(19) United States
(12) Patent Application Publication
(10) Pub. No.: US 2004/0127537 A1
(43) Pub. Date: Jul. 1, 2004

(54) Stable liquid parenteral parecoxib formulation

(76) Inventors: Yatin R. Gokarn, Thousand Oaks, CA (US); Richard Schartman, Wallingford, CT (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/464,015

(22) Filed: Jun. 18, 2003

Related U.S. Application Data
(60) Provisional application No. 60/391,714, filed on Jun. 26, 2002.

(51) Int. Cl. A61K 31/415

(52) U.S. Cl. 514/406

(57) ABSTRACT

A parenterally deliverable pharmaceutical composition comprises a water soluble parecoxib salt, in dissolved and/or solubilized form in a solvent liquid that comprises water and one or more nonaqueous solubilizer(s). Valdecoxib formed by conversion of parecoxib is solubilized by the nonaqueous solubilizer(s), which are substantially inert with respect to such conversion. The composition has parecoxib salt stabilizing means for inhibiting precipitation of parecoxib free acid. The composition is storage stable and is suitable for parenteral administration to treat a COX-2 mediated condition or disorder.

This application claims priority of U.S. provisional application Serial No. 60/391,714 filed on Jun. 26, 2002.

### FIELD OF THE INVENTION

The present invention relates to parenterally deliverable pharmaceutical compositions comprising the selective cyclooxygenase-2 (COX-2) inhibitory drug parecoxib, particularly to pharmaceutically acceptable salts of parecoxib, and more particularly parecoxib sodium. The invention also relates to processes for preparing such compositions, to therapeutic methods of use of such compositions and to use of such compositions in manufacture of medicaments.

### BACKGROUND OF THE INVENTION

Parenteral drug formulations have become a very important component in the arsenal of available drug delivery options, particularly for drugs having analgesic effect. Parenteral routes of administration, including subcutaneous, intramuscular and intravenous injection, offer numerous benefits over oral delivery in particular situations, for a wide variety of drugs. For example, parenteral administration of a drug typically results in attainment of a therapeutically effective blood serum concentration of the drug in a shorter time than is achievable by oral administration. This is especially true of intravenous injection, whereby the drug is placed directly in the bloodstream. Parenteral administration can also result in more predictable blood serum concentrations of a drug, because losses in the gastrointestinal tract due to metabolism, binding to food and other causes are eliminated. For similar reasons, parenteral administration often permits dose reduction. Parenteral administration is generally the preferred method of drug delivery in emergency situations, and is also useful in treating subjects who are uncooperative, unconscious, or otherwise unable or unwilling to accept oral medication.

If a parenteral drug formulation is to be prepared, it is preferable from patient convenience and safety standpoints that such a formulation be a ready-to-use formulation, i.e. one that does not require dilution or mixing immediately prior to use (as opposed to a reconstitutable formulation). Ready-to-use and dilutable liquid parenteral formulations can also be advantageous from a manufacturing standpoint by avoiding expensive lyophilization and/or other similar manufacturing steps.

U.S. Pat. No. 5,932,598 to Talley et al. discloses a class of water-soluble prodrugs of selective COX-2 inhibitory drugs, including the compound N-[[4-(5-methyl-3-phenylisoxazol-4-yl)phenyl]sulfonyl]propanamide, also referred to herein as parecoxib (I), and salts thereof, for example the sodium salt, referred to herein as parecoxib sodium. Parecoxib sodium is currently under development by Pharmacia Corp. for, inter alia, treatment of acute pain, for example post-surgical pain.

Parecoxib, which converts to the substantially water-insoluble selective COX-2 inhibitory drug valdecoxib following administration to a subject, itself shows weak in vitro inhibitory activity against both COX-1 and COX-2, while valdecoxib (II) has strong inhibitory activity against COX-2 but is a weak inhibitor of COX-1.

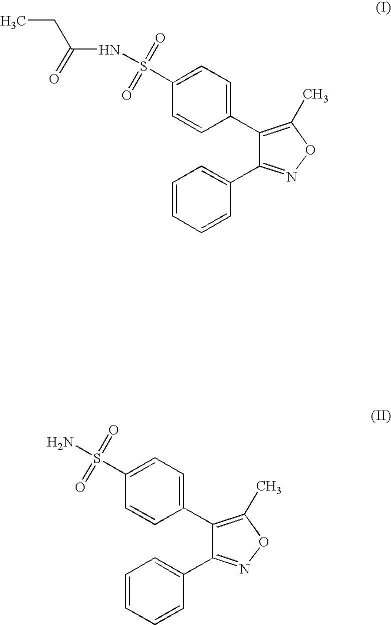

Because valdecoxib has very low solubility in water (about 10 μg/ml), it is not particularly well suited for formulation as a ready-to-use parenteral product. By contrast, because of the higher water solubility of parecoxib, particularly of salts such as parecoxib sodium, parecoxib has been proposed for liquid formulation. See Talley et al. (2000), J. Med. Chem. 43, 1661-1663. Unfortunately, attempts to formulate parecoxib sodium as a ready-to-use solution for injection have heretofore been complicated by the fact that parecoxib sodium, when in aqueous solution and especially in presence of certain excipients, is unstable, undergoing conversion to the relatively insoluble valdecoxib, which in turn precipitates out of solution to result in a formulation which is unsuitable for many forms of parenteral administration.

One potential solution to this problem is to provide a dry reconstitutable parecoxib formulation which is mixed with a liquid vehicle just prior to administration. However, in many situations it is particularly advantageous to provide a liquid formulation, more especially a ready-to-use formulation, as indicated above.

If a liquid parenterally deliverable formulation of parecoxib or a pharmaceutically acceptable salt thereof, particularly such a formulation that is ready-to-use, that is storage stable at room temperature could be prepared, a significant advance in treatment of COX-2 mediated conditions and disorders would result. This would be especially true for such conditions and disorders characterized by or accompanied by pain, particularly where rapid onset of pain relief is desired (as, for example, in migraine and other forms of acute and/or severe pain).

Patents and publications cited above are incorporated herein by reference.

### SUMMARY OF THE INVENTION

In one aspect, the present invention provides a parenterally deliverable pharmaceutical composition comprising parecoxib in a form of a water soluble parecoxib salt. The parecoxib salt is in dissolved and/or solubilized form in a solvent liquid that comprises water and one or more nonaqueous solubilizer(s). The nonaqueous solubilizer(s) are effective to solubilize valdecoxib that forms by conversion of parecoxib thereto, but are substantially inert with respect to such conversion, such that upon storage of the composition in a closed container maintained at 55° C. for 14 days, parecoxib constitutes at least about 95% of the total amount, expressed as parecoxib free acid equivalent, of parecoxib and valdecoxib in the composition. An important feature of the composition is that it has stabilizing means for inhibiting precipitation of parecoxib free acid.

In another aspect, the invention provides a parenterally deliverable pharmaceutical composition comprising a parecoxib component in a form of a water soluble parecoxib salt. The parecoxib salt is in dissolved and/or solubilized form in a solvent liquid that comprises:

(a) a water component;

(b) a nonaqueous solubilizer component effective to solubilize valdecoxib that forms by conversion of parecoxib thereto, the nonaqueous solubilizer component being substantially inert with respect to such conversion; and

(c) a parecoxib salt stabilizer component effective to inhibit precipitation of parecoxib free acid.

The nonaqueous solubilizer and parecoxib salt stabilizer components can be the same or different. Again, upon storage of the composition in a closed container maintained at 55° C. for 14 days, parecoxib constitutes at least about 95% of the total amount, expressed as parecoxib free acid equivalent, of parecoxib and valdecoxib in the composition.

In yet another aspect, the invention provides an article of manufacture comprising such a parenterally deliverable pharmaceutical composition in a sealed container.

Also provided is a method of treating a subject having a condition or disorder wherein treatment with a COX-2 inhibitory drug is indicated, the method comprising parenterally administering a therapeutically effective amount of a composition as described herein; and a method of use of such a composition in manufacture of a medicament useful in treating COX-2 mediated conditions and disorders by parenteral administration to a subject in need thereof.

Further provided is a process for preparing a parenterally deliverable pharmaceutical composition. The process comprises a step of combining in any order, with mixing:

(a) a parecoxib component in a form of a water soluble parecoxib salt;

(b) a water component;

(c) a nonaqueous solubilizer component effective to solubilize valdecoxib that forms by conversion of parecoxib thereto, said nonaqueous solubilizer component being substantially inert with respect to such conversion; and

(d) a parecoxib salt stabilizer component effective to inhibit precipitation of parecoxib free acid.

The nonaqueous solubilizer and parecoxib salt stabilizer components can be the same or different. The water, nonaqueous solubilizer and parecoxib salt stabilizer components form when mixed a solvent liquid wherein the parecoxib component is dissolved and/or solubilized. Upon storage of the composition in a closed container maintained at 55° C. for 14 days, parecoxib constitutes at least about 95% by weight of the total amount, expressed as parecoxib free acid equivalent, of parecoxib and valdecoxib in the composition.

Compositions of the invention can be provided at a parecoxib concentration suitable for parenteral delivery without mixing and/or dilution immediately prior to administration (i.e., “ready-to-use”), yet such compositions are surprisingly stable upon storage at room temperature and at refrigerated temperatures. Compositions of the invention, whether ready-to-use or requiring dilution prior to administration, can be prepared by processes that avoid the need for an expensive and time consuming lyophilization step. Other features of this invention will be in part apparent and in part pointed out hereinafter.

### BRIEF DESCRIPTION OF THE DRAWINGS

FIG. 1 is a graph showing stability of parecoxib sodium, as measured by amount of valdecoxib formed, in various solvent liquids, as described in Example 1.

FIG. 2 is a graph showing stability of parecoxib sodium, as measured by amount of valdecoxib formed, in solvent liquids containing water and various nonaqueous solubilizers, as described in Example 2.

FIG. 3 is a graph showing stability of parecoxib sodium, as measured by amount of valdecoxib formed, in solvent liquids comprising 35% water and 65% PEG of various average molecular weights, as described in Example 3.

FIG. 4 is a diagrammatic drawing of an illustrative article of manufacture of the invention, comprising a sealed vial having a parenterally deliverable pharmaceutical composition of a water soluble parecoxib salt in a solvent liquid occupying the fill volume, and a headspace overlying the composition filled with an oxygen limited microatmosphere.

### DETAILED DESCRIPTION OF THE INVENTION

Parecoxib Component

A water soluble parecoxib salt suitable for use in a composition of the invention can be prepared according to any process, for example processes known per se, including those described in above-cited U.S. Pat. No. 5,932,598. A “water soluble parecoxib salt” herein is one having solubility in water at 25° C. not less than about 10 mg/ml. Preferably, the salt has solubility in water at 25° C. not less than about 50 mg/ml, more preferably not less than about 100 mg/ml.

Pharmaceutically acceptable salts of parecoxib include metallic salts and organic salts. Preferred metallic salts include, but are not limited to, appropriate alkali metal (group Ia) and alkaline earth metal (group IIa) salts, and other physiologically acceptable metal salts. For example, such salts can be made from aluminum, calcium, lithium, magnesium, potassium, sodium and zinc. Preferred organic salts can be made from tertiary amines and quaternary ammonium salts, including without limitation tromethamine, diethylarnine, N,N′-dibenzylethylenediamine, chloroprocaine, choline, diethanolamine, ethylenediarnine, meglurnine (N-methylglucamine) and procaine. Selection from salts listed above can be based in part on water solubility.

Parecoxib sodium and parecoxib potassium are especially preferred parecoxib salts. The invention is described herein with particular reference to parecoxib sodium, but it will be understood that any other pharmaceutically acceptable salt of parecoxib can be used in conjunction with or in place of parecoxib sodium.

Where parecoxib salt and, specifically, parecoxib sodium concentrations, doses and amounts are set forth herein in mg/ml or other units, it will be further understood that such concentrations, doses and amounts are expressed as parecoxib free acid equivalent (“f.a.e.”), except where otherwise indicated or demanded by the context. Conversion to an equivalent concentration, dose or amount of any parecoxib salt can readily be calculated.

Similarly, an amount of valdecoxib expressed as parecoxib free acid equivalent is the amount of valdecoxib formed upon conversion thereto of the stated free acid equivalent amount of parecoxib.

Where the composition is intended for parenteral administration without further dilution, the parecoxib salt is typically present in a free acid equivalent amount of up to about 200 mg/ml, preferably about 1 to about 100 mg/ml, more preferably about 1 to about 90 mg/ml, and most preferably about 10 to about 50 mg/ml, for example about 20 to about 40 mg/ml. Where a composition of the invention is to be diluted prior to administration, higher parecoxib concentrations, for example about 200 to about 400 mg/ml, can be used.

The term “in dissolved and/or solubilized form” herein refers to that portion of the parecoxib that is present other than in solid form, for example other than as solid particulates of parecoxib salt or free acid suspended or otherwise dispersed in the solvent liquid. Typically that portion of the parecoxib described herein as “in dissolved and/or solubilized form” comprises parecoxib salt dispersed in molecular or ionic form in the solvent liquid.

Substantially all, for example at least about 95%, preferably at least about 98%, of the parecoxib present in a composition of the invention is in dissolved and/or solubilized form. Most preferably, no detectable amount of the parecoxib is in solid particulate form.

Solvent Liquid

A composition of the invention comprises a solvent liquid, one component of which is water. The solvent liquid further comprises at least one pharmaceutically acceptable nonaqueous solvent, co-solvent, and/or solubilizing agent (collectively referred to herein as solubilizers, whether or not their function in whole or in part is to solubilize a component of the composition). One or more additional pharmaceutically acceptable excipient(s) can also be present if desired in the solvent liquid, including a parecoxib salt stabilizing component as described below. Components of the solvent liquid are selected by type and amount so as to:

(a) dissolve and/or solubilize substantially all parecoxib salt present in the composition;

(b) not substantially enhance conversion of parecoxib to valdecoxib, for example by comparison with an otherwise similar solution of parecoxib salt in substantially pure water; and

(c) facilitate solubilization or dissolution of and/or inhibit precipitation of any valdecoxib which may form during storage of the composition by conversion of parecoxib; such solubilization or dissolution of valdecoxib by the solvent liquid or a component thereof being referred to herein as “resolubilization”.

The water component of the solvent liquid generally assures complete dissolution of the parecoxib salt; however it is important to select a nonaqueous solubilizer component that does not reduce solubility of the parecoxib salt to the point where precipitation of the salt or of parecoxib free acid occurs. Nonaqueous solubilizers useful herein are, as pointed out above, capable of solubilizing the small amount of valdecoxib that can form during storage, and are substantially inert with respect to conversion of parecoxib sodium to valdecoxib. The phrase “substantially inert” in this context means that parecoxib sodium present in a solvent liquid comprising the solubilizer should undergo substantially similar, or preferably less, conversion to valdecoxib by comparison with a solution of parecoxib sodium in substantially pure water or in 10 mM phosphate buffer at pH 8.1. Such a solubilizer can illustratively be selected according to Test I.

Test I

A. A candidate solubilizer, in substantially pure form or with up to about 5% water, by volume, is placed in a vessel at room temperature.

B. Parecoxib sodium is added to the solubilizer contained in the vessel in an amount equivalent to 40 mg/mil parecoxib free acid to form a test composition.

C. The test composition is stored in the vessel at 70° C. for 3 days.

D. Following such storage, concentration of valdecoxib formed in the test composition is measured.

E. If the amount of valdecoxib formed is not more than 4 mg/ml (equivalent to about 10% conversion of parecoxib), the candidate solubilizer is deemed suitable for use in a composition of the invention.

Preferably, the amount of valdecoxib measured in Step D is not more than about 2 mg/ml, more preferably not more than about 1.2 mg/ml.

Non-limiting examples of suitable nonaqueous solubilizers that can be present in the solvent liquid include polyethylene glycol (PEG), ethanol, dimethylacetamide (DMAC), propylene glycol, and mixtures thereof. It is preferred that the solvent liquid comprise at least one of PEG, DMAC and ethanol.

For purposes of establishing amounts or concentrations of a component in the solvent liquid, where the solvent liquid is part of a finished composition as provided herein, the solvent liquid is considered to comprise all components of the composition with the exception of parecoxib salt and free acid and its conversion product valdecoxib.

In a first preferred embodiment, the solvent liquid comprises DMAC and water. In this embodiment, DMAC is preferably present in the solvent liquid in an amount of about 0.01% to about 15%, preferably about 0.1% to about 10%, and more preferably about 0.5% to about 8%, by weight of the solvent liquid.

In a second preferred embodiment, the solvent liquid comprises ethanol and water. In this embodiment, ethanol is preferably present in the solvent liquid in an amount of about 1% to about 30%, preferably about 1% to about 25%, and more preferably about 1% to about 20%, by weight of the solvent liquid.

In a third preferred embodiment, the solvent liquid comprises PEG and water. PEGs for use in a solvent liquid of the invention have an average molecular weight of about 200 to about 6000, preferably 200 to about 1000, more preferably about 300 to about 900, and still more preferably about 400 to about 800. The terms “PEG 300”, “PEG 400” and “PEG 600” are used herein to refer to PEGs having average molecular weights of about 300, about 400 and about 600 respectively.

PEGs having average molecular weight greater than about 1000 are generally suitable only at relatively low concentrations in the solvent liquid, as at higher concentrations they give rise to unacceptably high viscosity. For PEG having average molecular weight of about 200 to about 1000, the weight ratio of PEG to water in a solvent liquid of this embodiment is preferably about 1:4 to about 4:1, more preferably about 1:3 to about 3:1, and most preferably about 1:2.5 to about 2.5:1. Alternatively, preferred concentrations of PEG in the solvent liquid are about 20% to about 80%, more preferably about 30% to about 70%.

Without being bound by theory, PEG is believed to be a particularly advantageous solubilizer because a PEG/water solvent liquid does not substantially promote conversion of parecoxib to valdecoxib, and because any valdecoxib which does form in such a solvent liquid tends to remain dissolved and/or solubilized therein. Thus, at least a substantial portion of valdecoxib formed upon storage of a composition of this embodiment does not precipitate as a particulate solid, which would be undesirable for parenteral administration.

PEGs of different average molecular weight differ in their inertness with respect to conversion of parecoxib to valdecoxib. Higher molecular weight PEGs are generally preferable in this regard. Good results are obtainable, for example, with PEG having an average molecular weight of about 300 to about 900, more preferably about 400 to about 800, e.g., about 600.

However, we have now surprisingly discovered a problem that is particularly troublesome in the case of PEG-containing parecoxib salt compositions. Such compositions tend to exhibit downward pH drift upon storage. Without being bound by theory, it is believed that such downward pH drift is due, at least in part, to oxidation of PEG and resultant formation of formic acid.

It has now been further discovered that parecoxib precipitates in free acid form as pH of the composition drifts below about 7.4. Presence of precipitated parecoxib free acid in a parenteral composition is undesirable as it can cause local pain and irritation upon injection. Therefore, at least where a composition of the invention comprises PEG, one or more of the means for stabilizing parecoxib salt described immediately below must be employed.

Parecoxib Salt Stabilizing Means

According to the invention, parecoxib salt is stabilized by means internal or external to the composition. Such means for stabilizing parecoxib salt generally act to inhibit, for example to slow, delay, reduce or prevent, precipitation of parecoxib in free acid form. It will be understood that the effectiveness of such means for stabilizing parecoxib salt, illustrative examples of which are individually described in further detail below, depend on, inter alia, composition of the particular solvent liquid, selection and amount of parecoxib salt, and desired final presentation of the composition

Limiting Effective Exposure to Oxygen

One class of suitable parecoxib salt stabilizing means, particularly for a PEG-containing composition of the invention, is a means for limiting effective exposure of the composition to oxygen. The term “limiting effective exposure of the composition to oxygen” includes placing the composition in contact with an oxygen-limited nmicroatmosphere and/or including in the composition one or more excipients or agents that mitigate potential deleterious effects of oxygen (e.g., formation of formic acid and consequent downward pH drift) on the composition. Limiting effective exposure of the composition to oxygen can be accomplished by one or more of the illustrative, non-limiting means described more fully immediately below.

One means for limiting effective exposure of the composition to oxygen is to place the composition in contact with an oxygen-limited microatmosphere in a sealed container. Such a container can have a substantial internal headspace occupied by a microatmosphere having low oxygen pressure. Alternatively, the container can have very little or no headspace, in which case effective exposure of the composition to oxygen is limited largely by the barrier effect provided by the sealed container itself The container and its contents form an article of manufacture and are a further embodiment of the invention.

Any suitable pharmaceutical container can be used to prepare an article of manufacture according to this embodiment. Such a container preferably encloses an amount of the composition corresponding to 1 to about 30 unit doses of parecoxib. The container can be a multi-dose container, enclosing an amount of the composition preferably corresponding to 2 to about 30, for example about 4 to about 20, unit doses. Alternatively, the container encloses an amount of the composition corresponding to a single unit dose. Such a single-dose article of manufacture has the further advantage of eliminating a measuring step before administration of the composition. Since compositions of the invention are desirable for parenteral administration, the container preferably is sufficient to maintain sterility of a composition contained therein. The container can also be used to facilitate direct administration (without need for transfer to another vessel or container) of a composition of the invention (e.g., a syringe). Non-limiting examples of suitable containers for an article of manufacture of this invention include vials of any shape and/or size, ampoules, syringes, packets, pouches, auto-injectors, etc. In one embodiment, the container further comprises means to protect the composition from exposure to light (e.g., amber glass walls). Non-limiting examples of a vial that can be used as a container in an article of manufacture of the present invention are described in U.S. Pat. No. 5,230,429 to Etheredge, incorporated herein by reference.

A composition of the invention can be sealed in a container in any suitable manner including but not limited to frictionally- and/or hermetically-induced seals. Such a seal can illustratively be provided by a stopper made of rubber or other polymeric material. A preferred seal comprises an inert coating, for example a coating of a fluoropolymer such as polytetrafluoroethylene (e.g., Teflon®) to prevent chemical interaction between the composition and the seal. The seal can illustratively be secured by a metal over-cap and/or an external cover (e.g., plastic) until use. Optionally, the seal can comprise at least one septum or thinner area of sealing material through which a needle can be inserted to extract the composition without cracking or breaking any glass or plastic portion of container wall. Regardless of what form of seal is used, such a seal should substantially inhibit movement of gas into or out of the container until the seal is penetrated for use of the composition present in the container.

Even where the composition is enclosed in a sealed container with an oxygen limited microatmosphere, effective exposure of the composition to oxygen is preferably further limited by one or more of the following means:

(a) a container size and/or shape that substantially maximizes fill volume and/or substantially minimizes headspace volume;

(b) low oxygen pressure in the headspace;

(c) use in the solvent liquid of water which has been purged of molecular oxygen; and

(d) use of a grade of PEG having a low peroxide content, for example not greater than about 1.5 meq/kg and preferably not greater than about 1.0 meq/kg.

The term “headspace” or “headspace volume” with respect to an article of manufacture of the invention refers to any interior volume of the container that is not occupied by, but is in contact with, the composition. Generally, the headspace volume is occupied by a gaseous medium. The term “fill volume” with respect to an article of manufacture of the invention refers to any interior volume of the container that is occupied by the composition.

The term “total volume” refers to the entire interior volume of the container and may also be referred to elsewhere as overflow volume; total volume generally equals the sum of the fill and headspace volumes. Importantly, for some containers, there can be a difference between the reported total volume and the actual total volume. The term “reported total volume” refers to the volume, for example as reported by the manufacturer of the container, that the container is designed to hold; this may be indicated, for example, by volume demarcations on container walls or by the name of the container (e.g., 2R container). However, some containers can actually hold a greater volume than the reported total volume. Unless the context demands otherwise, the term “total volume” herein refers to the actual total volume a container can hold, not the reported total volume.

Referring to FIG. 4, an illustrative article of manufacture of the invention comprises a vial 1 having a cap 2 comprising an airtight seal 3. Contained within the vial is a parenterally deliverable parecoxib composition as described herein occupying the fill space (i.e., the “fill volume”) 4. Overlying the composition is a headspace 5 containing a gaseous microatmosphere.

A suitable total volume for a container useful herein is determined by several factors. For example, various parenteral routes of administration differ with respect to practical minimum and maximum delivery volumes and accordingly dictate, in part, parecoxib concentration in a composition. Effective doses vary with respect to age of subject (e.g., infant versus adult) and with respect to therapeutic need. The composition volume necessary to achieve an effective unit dose depends upon the concentration of parecoxib in the composition. Accordingly, where parecoxib is present in a composition at 20 mg/ml and an effective parecoxib unit dose is 40 mg, 20 unit doses are provided by 40 ml and 1 unit dose by 2 ml of the composition. Similarly, where parecoxib is present in a composition at 50 mg/ml and an effective unit dose is 10 mg, 20 unit doses are provided by 4 ml and 1 unit dose by 0.2 ml of the composition. In these non-limiting examples, fill volumes to provide 1 to 20 unit doses vary from 0.2 to 40 ml. It will be understood that a particularly advantageous container volume depends on, inter alia, total volume of composition desired to be filled into the container and/or any other excipients present in the composition (e.g., antioxidants, buffering agents, etc.) which can limit effective exposure of the composition to oxygen or otherwise provide means for stabilizing parecoxib salt. Illustratively, where a container of standard size (e.g., 2R, 2 ml, 1 ml, etc.) is to be used, the container size selected is preferably the smallest available size that can hold the amount of composition desired to be filled therein. Alternatively, custom sized containers can be employed which are able to contain substantially no more volume of material than the volume of composition desired to be sealed therein.

While it is desirable to substantially minimize headspace volume, in practical terms it may not be necessary or feasible to eliminate all headspace from an article of manufacture of the invention. One of ordinary skill in the art will readily optimize headspace and fill volumes within the ranges provided herein and in view of other compositional attributes and desired shelf life of a final product. In a particularly preferred embodiment, the ratio of fill volume to headspace volume is not less than about 1:5 (i.e., not less than about 16% of the total container volume is occupied by a composition of the invention), preferably not less than about 1:3, more preferably not less than about 1:2, and still more preferably not less than about 1:1.

Another means for limiting effective exposure to oxygen of a composition in a sealed container comprises limiting oxygen pressure in the headspace of the container to less than about 5% (i.e., about 0.05 atm), preferably less than about 3%, more preferably less than about 2.5%, even more preferably less than about 2%, and most preferably less than about 1%.

Importantly, it will be understood that a relationship between suitable headspace volume on the one hand and suitable oxygen pressure on the other, exists. One of ordinary skill in the art will be able to optimize these factors within the ranges provided herein (and in light of other composition characteristics) in order to obtain a composition exhibiting suitable stability over a desired shelf life. As headspace volume increases, it becomes more important to provide a relatively low oxygen pressure in the headspace. Thus, where the ratio of fill volume to headspace volume is low (e.g., about 1:5), oxygen pressure in the headspace is also preferably at the low end of the range provided herein (e.g., less than about 1%). By contrast, where the ratio of fill volume to headspace volume is toward the higher end of the range provided herein (e.g., 1:1 or greater), higher oxygen pressure in the headspace may be acceptable while still providing suitable product stability for a particular desired shelf life.

Illustratively, where a container has a total volume of about 4 ml and a composition of the invention sealed therein occupies a fill volume of about 1 ml, oxygen pressure in the headspace is preferably towards the low end of the range provided herein, for example not more than about 2% and preferably not more than about 1%. On the other hand, where 1 ml of the same composition is to be sealed into a container having a total volume of about 1.25 ml, oxygen pressure in the headspace can be higher, for example about 2% to about 3%.

Oxygen pressure in a container headspace of an article of manufacture of the invention can be limited in any suitable manner, illustratively by placing nitrogen and/or a noble gas (collectively referred to herein as “inert gases”) in the container headspace. In this embodiment, the headspace volume preferably comprises one or more inert gases selected from the group consisting of nitrogen, helium, neon and argon. An illustrative process for placing such a gas in a container headspace is described in U.S. Pat. No. 6,274,169 to Abrahamson et al., incorporated herein by reference.

One way to ensure low oxygen pressure in the headspace is to prepare, fill and seal the container under an atmosphere of inert gas and/or to flush the container headspace with inert gas after filling, illustratively using parallel in-line flushing. An inert gas atmosphere can illustratively be provided using a zero oxygen tunnel commercially available from Modified Atmosphere Packaging Systems of Des Plaines, Ill., or by using a nitrogen or noble gas atmosphere glove bag.

Oxygen pressure in the headspace of an article of manufacture of the invention can be measured according to any suitable method, illustratively using an electrochemical cell (e.g., Checkmate 9900 oxygen analyzer), Raman Spectroscopy, and/or photoelectric systems for detecting elemental composition of a medium. Non-limiting examples of suitable methods for detection of oxygen in a container headspace are described in detail in the following publications, incorporated herein by reference.

International Patent Publication No. WO 96/02835.

Powell et al. (1986), Analytical Chemistry, 58, 2350-2352.

Bailey et al. (1980), Journal of the Parenteral Drug Association, 34(2), 127-133.

Yet another suitable means for limiting effective exposure of a composition, particularly a PEG-containing composition, of the invention to oxygen, and thereby providing parecoxib salt stabilizing means, comprises one or more pharmaceutically acceptable antioxidants, preferably free-radical scavenging antioxidants, as a component of the solvent liquid. Non-limiting illustrative examples of suitable antioxidants include α-tocopherol (vitamin E), ascorbic acid (vitamin C) and salts thereof including sodium ascorbate and ascorbic acid palmitate, butylated hydroxyanisole (BHA), butylated hydroxytoluene (BHT), fumaric acid and salts thereof, hypophosphorous acid, malic acid, methionine, alkyl gallates, for example propyl gallate, octyl gallate and lauryl gallate, sodium sulfite, sodium bisulfite and sodium metabisulfite. Preferred free radical-scavenging antioxidants are alkyl gallates, vitamin E, BHA, BHT, ascorbate and methionine, more especially BHA, ascorbate and methionine. Preferably, an antioxidant is selected that is substantially soluble in the particular solvent liquid employed and does not result in changes to the composition which are detectable by unaided sensory organs (e.g., color or odor changes). BHA is an illustrative preferred antioxidant for use in a composition of the invention. If included, one or more antioxidants are preferably present in a composition of the invention in a total antioxidant amount of about 0.001% to about 5%, preferably about 0.001% to about 2.5%, and still more preferably about 0.001% to about 1%, by weight.

In addition to or in place of means for limiting effective exposure of a composition, particularly a PEG-containing composition, to oxygen, another suitable parecoxib salt stabilizing means that can be used is a pH controlling means for maintaining the composition at a pH not lower than about 7.4. An example of a pH controlling means is a buffer. Unexpectedly, we have now discovered that in a composition that comprises a solvent liquid having a relatively high PEG concentration (e.g., not less than about 50% by weight of the solvent liquid), parecoxib sodium itself is an excellent buffer. Therefore, in one embodiment, a suitable pH controlling means comprises a solvent liquid having a PEG concentration therein which is effective to buffer the composition at a pH not lower than about 7.4. The term “effective to buffer the composition” in the present context means that such a PEG concentration provides a composition less susceptible to pH reduction upon titration with acid, by comparison with an otherwise similar composition having not more than 40% PEG by weight of the solvent liquid. In this embodiment, the PEG concentration in the solvent liquid is preferably not less than about 50%, more preferably not less than about 52.5%, and still more preferably not less than about 55%.

Additional buffering agents in addition to the parecoxib salt itself can be present if desired, but are not presently believed to be necessary. If an additional buffer is to be used, it should exhibit good solubility (e.g., not less than about 0.01 M at 25° C.) in the particular solvent liquid being employed, should result in minimal or substantially no changes to the composition which are detectable by unaided sensory organs (e.g., color or odor changes), and should not substantially enhance conversion of parecoxib to valdecoxib. Non-limiting illustrative examples of pharmaceutically acceptable buffering agents include phosphate, 2-amino-2-(hydroxymethyl)-1,3-propanediol (“tris”), ascorbate and maleate buffers. If included, one or more buffering agents are preferably present at a concentration of about 1 to about 50 mM and preferably about 1 to about 25 mM in the composition. Where the buffer includes a cation, it is preferred to match the cation to that of the parecoxib salt; thus, for example, in the case of a parecoxib sodium composition, sodium ascorbate or sodium maleate would be suitable buffers.

Yet another means for stabilizing parecoxib salt in a PEG-containing composition is a metal sequestering agent or chelating agent as a component of the solvent liquid. Without being bound by theory, it is believed that such agents prevent oxidation of PEG and thereby inhibit formation of formic acid and reduce tendency for downward pH drift. Suitable sequestering agents may be selected from ethylene diamine tetraacetic acid (EDTA), potassium polyphosphate, sodium polyphosphate, potassium metaphosphate, sodium metaphosphate, dimethylglyoxirne, 8-hydroxyquinoline, nitrilotriacetic acid, dihydroxyethylglycine, gluconic acid, citric acid and tartaric acid. These are illustratively used in an amount of about 0.01% to about 2% by weight of the solvent liquid.

In preferred embodiments, one or more means for stabilizing parecoxib salt are selected such that following storage of a composition in a closed container maintained under ambient conditions for a period of about 1 month, more preferably about 2 months, still more preferably about 6 months, and even more preferably about 1 year, the composition has a pH not less than about 7.4, preferably not less than about 7.7, and more preferably not less than about 8.0. Where a composition of the invention comprises PEG, pH of the composition is preferably controlled between about 7.4 and about 12, more preferably between about 7.7 and about 10, and still more preferably between about 8.0 and about 9.5.

Conversion of Parecoxib to Valdecoxib During Storage

As described above, selection of excipients is critical to prevent excessive conversion of parecoxib to valdecoxib during storage. A composition of the invention exhibits less than about 5% conversion of parecoxib to valdecoxib when stored in a closed container at the elevated temperature of 55° C. for 14 days; in other words, the amount of parecoxib remaining after such a storage treatment is at least about 95% of that initially present. Since essentially all conversion of parecoxib is to valdecoxib, another way of stating the same thing is that the amount of parecoxib present after such a storage treatment is at least about 95% of the total amount, expressed as parecoxib free acid equivalent, of parecoxib and valdecoxib present.

In preferred embodiments, the composition can be stored in a closed container maintained at 55° C. for a period of up to about 30 days, more preferably up to about 90 days, and still more preferably up to about 180 days, without the amount of parecoxib falling below about 95%, preferably about 96%, more preferably about 97%, and still more preferably about 98% of parecoxib originally present in the composition.

Preferably, upon storage of a composition of the invention in a closed container maintained under ambient conditions for a period of about 1 month, preferably about 2 months, more preferably about 6 months, still more preferably about 1 year, and even more preferably about 2 years, parecoxib constitutes at least about 92.5%, preferably at least about 95%, more preferably at least about 97%, and still more preferably at least about 98%, by weight, of parecoxib originally present in the composition. Again, one skilled in the art can readily estimate the amount of parecoxib originally present as the sum of parecoxib and valdecoxib amounts, expressed as parecoxib free acid equivalent, present at any later time. No serious error results from simply adding parecoxib and valdecoxib amounts expressed by weight.

Therapeutic use of Compositions of the Invention

Compositions of the invention are useful in treatment and prevention of a very wide range of disorders mediated by-COX-2, including but not restricted to disorders characterized by inflammation, pain and/or fever. Such compositions are especially useful as anti-inflammatory agents, such as in treatment of arthritis, with the additional benefit of having significantly less harmful side effects than compositions of conventional NSAIDs that lack selectivity for COX-2 over COX-1. In particular, compositions of the invention have reduced potential for gastrointestinal toxicity and gastrointestinal irritation, including upper gastrointestinal ulceration and bleeding, by comparison with compositions of conventional NSAIDs. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinernia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendonitis, bursitis, allergic neuritis, cytomegalovirus infection, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, scleroderma, rheumatic fever, type 1 diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic disorders, including without limitation inflammatory disorders such as endophthalmitis, episcleritis, retinitis, iriditis, cyclitis, choroiditis, keratitis, conjunctivitis and blepharitis, inflammatory disorders of more than one part of the eye, e.g., retinochoroiditis, iridocyclitis, iridocyclochoroiditis (also known as uveitis), keratoconjunctivitis, blepharoconjunctivitis, etc.; other COX-2 mediated retinopathies including diabetic retinopathy; ocular photophobia; acute trauma of any tissue of the eye including postsurgical trauma, e.g., following cataract or corneal transplant surgery; postsurgical ocular inflammation; intraoperative miosis; corneal graft rejection; ocular, for example retinal, neovascularization including that following injury or infection; macular degeneration; cystoid macular edema; retrolental fibroplasia; neovascular glaucoma; and ocular pain.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, bums, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemangiomas, angiofibroma of the nasopharynx and avascular necrosis of bone; and disorders of the female- reproductive system such as endometriosis.

Such compositions are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in subjects at risk of FAP.

Such compositions inhibit prostanoid-induced smooth muscle contraction by inhibiting synthesis of contractile prostanoids and hence can be of use in treatment of dysmenorrhea, premature labor, asthma and cosinophil-related disorders. They also can be of use for decreasing bone loss particularly in postmenopausal women (i.e., treatment of osteoporosis), and for treatment of glaucoma.

Preferred uses for compositions of the present invention are for treatment of rheumatoid arthritis and osteoarthritis, for pain management generally (particularly post-oral surgery pain, post-general surgery pain, post-orthopedic surgery pain, and acute flares of osteoarthritis), for prevention and treatment of headache and migraine, for treatment of Alzheimer's disease, and for colon cancer chemoprevention

Besides being useful for human treatment, compositions of the invention are useful for veterinary treatment of companion animals, exotic animals, farm animals, and the like, particularly mammals. More particularly, compositions of the invention are useful for treatment of COX-2 mediated disorders in horses, dogs and cats.

The present invention is further directed to a therapeutic method of treating a condition or disorder where treatment with a COX-2 inhibitory drug is indicated, the method comprising parenteral administration of a composition of the invention to a subject in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely and can therefore deviate from the preferred dosage regimens set forth herein.

Initial treatment can begin with a dose regimen as indicated herein. Treatment is generally continued as necessary over a period of several weeks to several months or years until the condition or disorder has been controlled or eliminated. Subjects undergoing treatment with a composition of the invention can be routinely monitored by any of the methods well known in the art to determine effectiveness of therapy. Continuous analysis of data from such monitoring permits modification of the treatment regimen during therapy so that optimally effective doses are administered at any point in time, and so that the duration of treatment can be determined. In this way, the treatment regimen and dosing schedule can be rationally modified over the course of therapy so that the lowest amount of the composition exhibiting satisfactory effectiveness is administered, and so that administration is continued only for so long as is necessary to successfully treat the condition or disorder.

The term “parenteral administration” herein encompasses injection and/or infusion of a composition by means other than through the gastrointestinal tract such as into or through the skin of a subject, and includes intradermal, subcutaneous, intramuscular, intravenous, intramedullary, intra-articular, intrasynovial, intraspinal, intrathecal and intracardiac administration. Any known device useful for parenteral injection or infusion of drugs can be used to effect such administration.

The term “effective dose” herein means a dose that is deemed to be effective for a medical purpose (e.g. prophylactic or therapeutic) and will vary depending upon many factors. Such non-limiting factors include route and frequency of administration and medical purpose.

The term “unit dose” herein means an amount of parecoxib or of a composition comprising parecoxib, such amount being suitable for delivery in a single administration event.

It has been found that parecoxib, when administered parenterally to a human subject, is rapidly and completely converted to valdecoxib. Therefore, even where rapid onset of therapeutic effect is desired, a therapeutically effective dose of parecoxib, for example in the form of parecoxib sodium, is one that is equal to a therapeutically effective dose of valdecoxib administered orally. The term “equal” in this context means equal in molar amount or in absolute amount (i.e., in weight). Based on molecular weights, complete conversion of 1 mg parecoxib produces about 0.85 mg valdecoxib. For practical purposes, no great error arises from considering 1 mg parecoxib to be equivalent to 1 mg valdecoxib.

Thus according to an embodiment of the present invention, a method is provided for treatment of a COX-2 mediated disorder in a human subject comprising parenterally administering a composition as described herein to the subject at a parecoxib dosage equal to a therapeutically effective dosage of valdecoxib. Preferably, the parecoxib salt is administered in a daily dosage amount of about 1 mg to about 150 mg. More preferred daily dosage amounts are about 5 mg to about 120 mg, more preferably about 10 mg to about 100 mg, and still more preferably about 15 mg to about 50 mg, for example about 20 mg or about 40 mg, parecoxib.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokiin-1 receptor antagonists and sodium channel blockers, among others.

Preferred combination therapies comprise use of a composition of the invention with one or more compounds selected from aceclofenac, acemetacin, ε-acetamidocaproic acid, acetaminophen, acetaminosalol, acetanilide, acetylsalicylsalicylic acid, S-adenosyhnethionine, alclofenac, alfentanil, allylprodine, alminoprofen, aloxiprin, alphaprodine, aluminum bis(acetylsalicylate), amfenac, amino chlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, aspirin, balsalazide, bendazac, benorylate, benoxaprofen, benzpiperylon, benzydamine, benzylmnorphine, berberine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butorphanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac, difenamizole, difenpiramide, diflunisal, dihydrocodeine, dihydrocodeinone enol acetate, dihydromorphine, dihydroxyaluminum acetylsaiicylate, dimenoxadol, dimepheptanol, dimethylthiambutene, dioxaphetyl butyrate, dipipanone, dipyrocetyl, dipyrone, ditazol, droxicam, emorfazone, enfenamnic acid, epirizole, eptazocine, etanercept, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, infliximab, interleukin-10, isofezolac, isoladol, isomethadone, isonixin, isoxepac, isoxicam, ketobemidone, ketoprofen, ketorolac, p-lactophenetide, lefetamine, levorphanol, lexipafant, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozim, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamnide, salicin, salicylamide, salicylamide o-acetic acid, salicylsulfuric acid, salsalate, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen, ziconotide and zomepirac (see The Merck Index, 13th Edition (2001), Therapeutic Category and Biological Activity Index, lists therein headed “Analgesic” , “Anti-inflammatory” and “Antipyretic”).

Particularly preferred combination therapies comprise use of a composition of the invention with an opioid compound, more particularly where the opioid compound is codeine, meperidine, morphine or a derivative thereof.

The drug being used in combination therapy with parecoxib or a composition of the invention can be administered by any route, including parenterally, orally, topically, etc.

### EXAMPLES

The following examples are provided for illustrative purposes only and are not to be construed to limit the invention in any way.

### Example 1

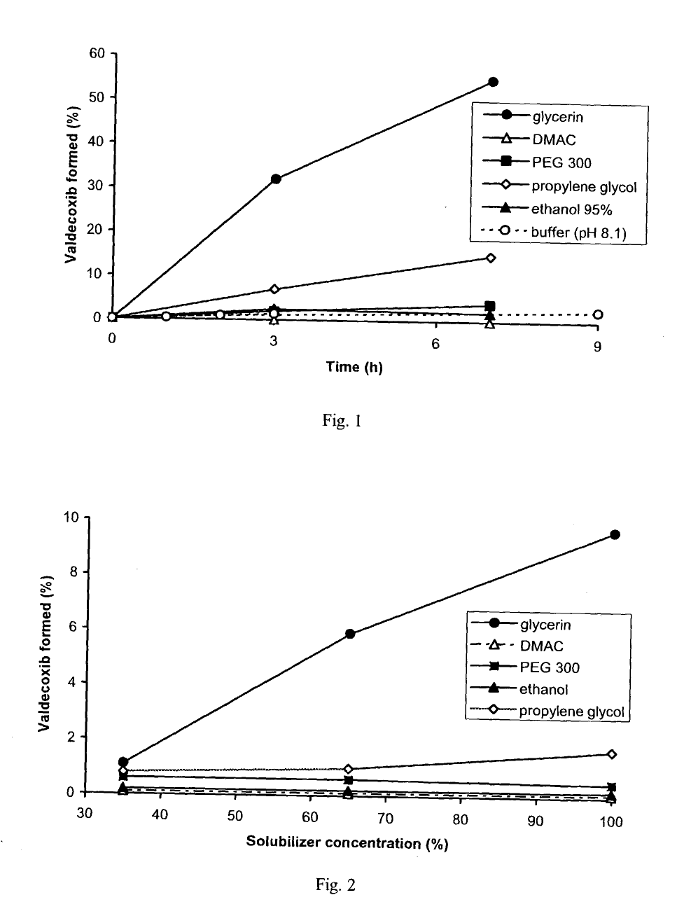

Stability of parecoxib sodium (42.36 mg/ml, equivalent to 40 mg/ml parecoxib free acid) dissolved in various candidate solubilizers was determined over a period of 7 days at 70° C. Formulations were prepared and sealed in Type I vials. Concentration of valdecoxib formed in presence of each solubilizer was measured using HPLC. Valdecoxib formed was calculated as a percentage of parecoxib initially present. As shown in FIG. 1, less valdecoxib was formed in presence of PEG 300, DMAC, 95% ethanol, or 10 mM phosphate buffer (pH 8.1) than in presence of propylene glycol. In presence of glycerin, conversion of parecoxib to valdecoxib was very rapid, demonstrating that glycerin is not an acceptable solubilizer for use in a composition of the present invention.

### Example 2

Stability of parecoxib sodium in several solvent liquids (comprising different solubilizers at 35% or 65% concentration in water or in absence of water) was determined over a period of 3 days at 55° C. The procedure was similar to that of Example 1. Valdecoxib formed was calculated as a percentage of parecoxib initially present. As shown in FIG. 2, parecoxib was stable in solvent liquids comprising PEG 300, ethanol or DMAC, with or without water, but less so in propylene glycol containing solvent liquids. Parecoxib exhibited significant conversion to valdecoxib in solvent liquids comprising glycerin.

### Example 3

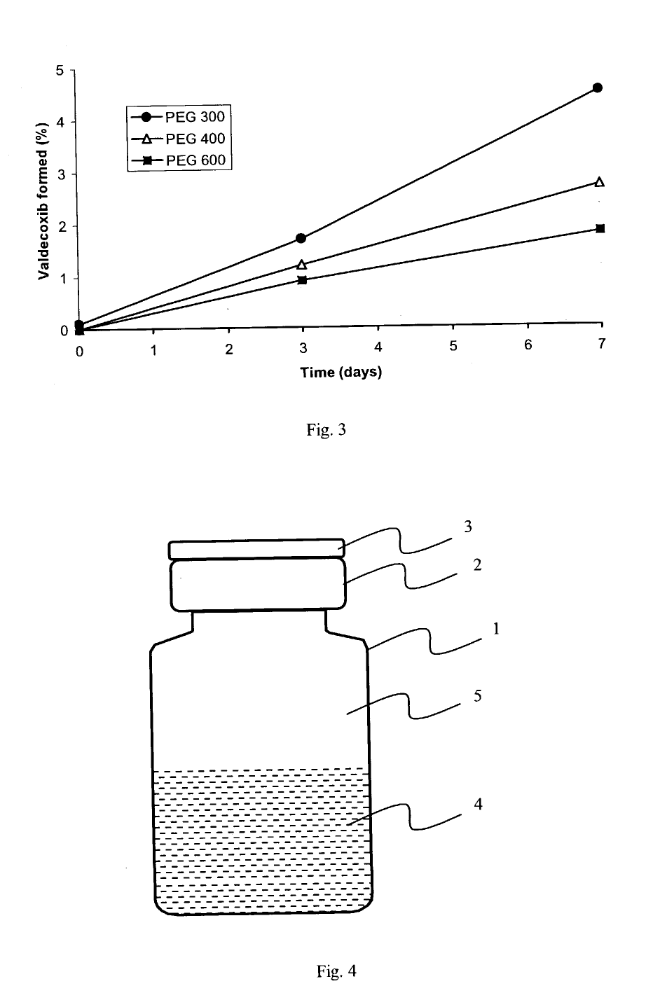

Stability of parecoxib sodium in three different PEG-containing solvent liquids (65% PEG 300 in water, 65% PEG 400 in water, or 65% PEG 600 in water) was assessed at 70 ° C. over a period of 7 days. The procedure was similar to that of Example 1. Valdecoxib formed was calculated as a percentage of parecoxib initially present. As shown in FIG. 3, parecoxib was acceptably stable in each solvent liquid tested, but less valdecoxib formed in the solvent liquids containing PEG 600 or PEG 400 than in the solvent liquid containing PEG 300.

### Example 4

Nine liquid parecoxib sodium formulations, LF1-LF9, were prepared having compositions as shown in Table 1. Each formulation had a solvent liquid comprising PEG and water for injection (WFI), with parecoxib sodium dissolved therein at 84.72 mg/ml (80 mg/ml parecoxib free acid equivalent) and was sterile filtered and placed in a type I glass vial with a reported total volume of 2 ml (actual total volume of 3.224 ml). Each vial was flushed with nitrogen gas prior to stoppering.

TABLE 1
Composition of parecoxib liquid formulations of Example 4

| | | | | | | | | |
|---|---|---|---|---|---|---|---|---|
| LF1 | LF2 | LF3 | LF4 | LF5 | LF6 | LF7 | LF8 | LF9 |

parecoxib sodium (mg/ml, free acid equivalent)

| | | | | | | | | |
|---|---|---|---|---|---|---|---|---|
| 80 | 80 | 80 | 80 | 80 | 80 | 80 | 80 | 80 |

components of solvent liquid (% by weight)

| | | | | | | | | | |
|---|---|---|---|---|---|---|---|---|---|
| PEG | 65 | 55 | 45 | | | | | | |
| 600 | | | | | | | | | |
| PEG | | | | 65 | 55 | 45 | | | |
| 400 | | | | | | | | | |
| PEG | | | | | | | 65 | 55 | 45 |
| 300 | | | | | | | | | |
| WFI | 35 | 45 | 55 | 35 | 45 | 55 | 35 | 45 | 55 |

Multiple vials of each of parecoxib sodium liquid formulations LF1, LF2, LF3, LF5, LF7, LF8 and LF9 were individually stored at 30, 40, 55 or 70° C. over a period of 91 days during which time parecoxib sodium stability was assessed at intervals by determining amount of valdecoxib formed. Valdecoxib was measured by HPLC and was calculated as a percentage of parecoxib initially present.

TABLE 2
Valdecoxib formed in formulations of Example 4

| Temp. | Time | Valdecoxib formed (%) | | | | | | |
|---|---|---|---|---|---|---|---|---|
| (° C.) | (days) | LF1 | LF2 | LF3 | LF5 | LF7 | LF8 | LF9 |
| 70 | 3 | 0.94 | 1.00 | 1.64 | 1.21 | 0.95 | 1.63 | 1.56 |
| 70 | 7 | 2.08 | 2.12 | 2.26 | 2.85 | 3.43 | 3.60 | 3.73 |
| 70 | 14 | 4.03 | 4.36 | 4.64 | 5.65 | 6.86 | 6.94 | 7.07 |
| 55 | 7 | 0.61 | 0.60 | 0.70 | 0.74 | 1.07 | 1.03 | 1.02 |
| 55 | 14 | 1.22 | 1.11 | 1.18 | 1.44 | 2.05 | 2.01 | 1.97 |
| 55 | 28 | 2.17 | 2.20 | 2.26 | 2.86 | 3.97 | 3.92 | 3.83 |
| 55 | 42 | 3.20 | 3.14 | 3.38 | 4.31 | | | |
| 55 | 56 | 3.97 | 4.10 | 4.42 | 5.65 | | | |
| 40 | 14 | 0.29 | 0.32 | 0.34 | 0.38 | 0.57 | 0.58 | 0.57 |
| 40 | 28 | 0.55 | 0.58 | 0.65 | 0.70 | 1.04 | 1.05 | 1.04 |
| 40 | 42 | 0.76 | 0.80 | 0.87 | 1.02 | 1.46 | 1.49 | 1.45 |
| 40 | 56 | 1.00 | 1.05 | 1.12 | 1.36 | | | |
| 40 | 91 | 1.58 | 1.67 | 1.78 | 2.21 | | | |
| 30 | 28 | 0.21 | 0.24 | 0.27 | 0.28 | 0.42 | 0.45 | 0.45 |
| 30 | 56 | 0.38 | 0.44 | 0.51 | 0.52 | | | |
| 30 | 91 | 0.58 | 0.65 | 0.69 | 0.81 | | | |

As hown in Table 2, each of liquid formulations LF1, LF2, LF3, LF5, LF7, LF8 and LF9 exhibited valdecoxib formation in an amount less than 5% of parecoxib initially present, when stored at 55° C. for 14 days. This is evidence of acceptable stability of parecoxib in these formulations. Data from this study suggest that parecoxib sodium stability increases with increasing PEG molecular weight. Compare, for example, valdecoxib formed in formulations LF2 (55% PEG 600), LF5 (55% PEG 400) and LF8 arecoxib sodium liquid formulations, LF1 LF15, were prepared having (55% PEG 300).

### Example 5

Six parecoxib sodium liquid formulations, LF10-LF15, were prepared having composition as shown in Table 3. Each formulation had a solvent liquid comprising PEG 600 and water for injection (WFI), with parecoxib sodium dissolved therein at a concentration of 42.36 mg/ml (40 mg/ml parecoxib free acid equivalent). Aliquots of each formulation (2 ml) were sterile filled into Type I vials and the vials were sealed with stoppers. Nitrogen gas was flushed into the headspaces of each vial containing formulations LF10-LF12 only.

TABLE 3
Composition of parecoxib liquid formulations of Example 5

| | | | | | |
|---|---|---|---|---|---|
| LF10 | LF11 | LF12 | LF13 | LF14 | LF15 |

parecoxib sodium (mg/ml, free acid equivalent)

| | | | | | |
|---|---|---|---|---|---|
| 40 | 40 | 40 | 40 | 40 | 40 |

components of solvent liquid (% by weight)

| | | | | | | |
|---|---|---|---|---|---|---|
| PEG | 45 | 55 | 65 | 45 | 55 | 65 |
| 600 | | | | | | |
| WFI | 55 | 45 | 35 | 55 | 45 | 35 |

nitrogen flush

| | | | | | |
|---|---|---|---|---|---|
| yes | yes | yes | no | no | no |

Formulations LF10-LF15 were maintained at 40, 55, and 70° C. for up to 28 days. Stability of each formulation was assessed using HPLC by measuring valdecoxib formation. Data are shown in Table 4.

TABLE 4
Valdecoxib formed in formulations of Example 5

| | | Valdecoxib Formed (%) | | | | | |
|---|---|---|---|---|---|---|---|
| | | Nitrogen flush | | | No nitrogen flush | | |
| Temp. (° C.) | Time (days) | LF10 | LF11 | LF12 | LF13 | LF14 | LF15 |
| 70 | 3 | 1.1 | 0.99 | 0.94 | 1.4 | 1.2 | 1.5 |
| 70 | 7 | 2.3 | 2.0 | 2.0 | 2.9 | 2.6 | 3.0 |
| 70 | 14 | 5.5 | 4.8 | 4.8 | 5.4 | 4.8 | 5.3 |
| 55 | 7 | 0.62 | 0.62 | 0.78 | 0.94 | 0.80 | 0.91 |
| 55 | 14 | 1.3 | 1.1 | 1.2 | 1.8 | 1.6 | 1.9 |
| 55 | 28 | 2.3 | 2.1 | 2.1 | 3.0 | 2.8 | 3.4 |
| 40 | 14 | 0.40 | 0.33 | 0.37 | 0.52 | 0.50 | 0.40 |
| 40 | 28 | 0.70 | 0.61 | 0.62 | 0.97 | 0.81 | 0.80 |

These data show that formulations placed in a vial with nitrogen flush exhibit decreased valdecoxib formation by comparison with formulations filled into vials without nitrogen flush.

### Example 6

Individual solubility of thirteen buffers was determined in a solvent liquid containing 65% PEG 600 and 35% water. Those buffers exhibiting a room temperature solubility of 0.01 M or greater, as shown in Table 5, were further evaluated as described immediately below.

TABLE 5
Solubility of various buffers in a PEG 600/water solvent liquid

| Buffer | Solubility > 0.01 M |
|---|---|
| sodium acetate | yes |
| L-arginine | yes |
| sodium ascorbate | yes |
| aspartic acid | no |
| sodium citrate | no |
| glycine | yes |
| histidine | no |
| sodium lactate | yes |
| sodium maleate | yes |
| sodium phosphate | no |
| sodium succinate | no |
| sodium tartrate | yes |
| tris | yes |

Selected buffers were-titrated in formulations comprising a 55% or 65% by weight PEG 600 in water for injection solvent liquid with parecoxib sodium dissolved therein at a concentration of 42.36 mg/ml (40 mg/ml parecoxib free acid equivalent), having pH of 8.5. The amount of titrant used to titrate each parecoxib sodium/buffer solution was compared with that required to titrate a parecoxib solution without buffer. Percent improvement in buffer capacity was determined by comparing the amount of acid required to adjust pH from 8.5 to 7.4 in the presence versus absence of buffer. Data are shown in Table 6.

TABLE 6
Improvement in buffer capacity due to added buffer

| PEG 600/WFI | | Improvement in |
|---|---|---|
| (% by weight) | Buffer | buffer capacity |
| 65/35 | tris | 63% |
| 55/45 | tris | 229% |
| 65/35 | sodium acetate | negligible |
| 55/45 | sodium acetate | negligible |
| 65/35 | L-arginine | negligible |
| 65/35 | sodium maleate | 21% |
| 65/35 | sodium lactate | negligible |
| 65/35 | glycine | negligible |
| 55/45 | glycine | negligible |
| 65/35 | sodium ascorbate | 14-37%¹ |

¹Broad range suggests that repeater pipette misfired.

These data show that tris, maleate and ascorbate buffers further improved buffer capacity of the parecoxib sodium formulations tested. The large improvement in buffer capacity seen for the tris buffer in the 55% PEG formulation by comparison with the 65% PEG-in-water formulation is consistent with observations that parecoxib sodium, itself, is an excellent buffer in more highly concentrated PEG solutions but has weaker buffering effect in less concentrated PEG solutions.

### Example 7

Eight parecoxib sodium liquid formulations, LF16-LF23, were prepared having compositions as shown in Table 7. Each formulation comprised PEG 600, water for injection, buffer, and parecoxib sodium at a concentration of 42.36 mg/ml (40 mg/ml parecoxib free acid equivalent). Aliquots (1.4 ml) of each formulation were filled into 2R (actual total volume 4.099 ml) vials; headspaces in vials containing formulations LF 16-LF19 were flushed with nitrogen (so that each-headspace comprised <5% oxygen) while headspaces in vials containing formulations LF20-LF23 were not flushed with nitrogen.

TABLE 7
Composition of parecoxib liquid formulations of Example 7

| | | PEG | | | |
|---|---|---|---|---|---|
| | Parecoxib | 600/WFI | | | |
| | Na (mg/ml | (% by | | Conc. | Nitrogen |
| Formulation | f.a.e.) | weight) | Buffer | (mM) | flush |
| LF16 | 40 | 65/35 | tris | 40 | yes |
| LF17 | 40 | 65/35 | sodium maleate | 40 | yes |
| LF18 | 40 | 65/35 | tris | 10 | yes |
| LF19 | 40 | 65/35 | sodium maleate | 10 | yes |
| LF20 | 40 | 65/35 | tris | 40 | no |
| LF21 | 40 | 65/35 | sodium maleate | 40 | no |
| LF22 | 40 | 65/35 | tris | 10 | no |
| LF23 | 40 | 65/35 | sodium maleate | 10 | no |

Formulation LF20 was maintained at 55° C. for 1 week. Formulations LF21 and LF23 were maintained at 55° C. for 4 weeks. Amount of valdecoxib formed in each of Formulations LF20, LF21 and LF23 after storage was assessed using HPLC; data are shown in Table 8.

TABLE 8
Valdecoxib formed in formulations of Example 7

| Formulation | Time (weeks) | Valdecoxib formed (%) |
|---|---|---|
| LF20 | 1 | 3.0 |
| LF21 | 4 | 2.1 |
| LF23 | 4 | 2.6 |

### Example 8

Four parecoxib liquid formulations, LF25-LF29, were prepared having compositions as shown in Table 9. Initial pH of each formulation was 8.5.

TABLE 9
Composition (mg/ml) of formulations of Example 8

| Formulation | LF25 | LF26 | LF27 | LF28 |
|---|---|---|---|---|
| parecoxib sodium | 42.36 | 42.36 | 42.36 | 42.36 |
| PEG 600 | 698 | 698 | 698 | 698 |
| sodium maleate | — | 1.6 | — | 1.6 |
| BHA | — | — | 0.025 | 0.025 |
| water for injection | q.s. | q.s. | q.s. | q.s. |
| Total volume (ml) | 1 | 1 | 1 | 1 |

Aliquots (0.7 ml or 1.2 ml) of each of formulations LF25 -LF28 were filled into 2 ml vials (actual total volume 3.224 ml). The headspace of each vial was filled with nitrogen gas using a lyophilizer, evacuating each vial with 10 -50 μm pressure and breaking the vacuum with nitrogen gas. This resulted in an oxygen pressure of about 2% in the headspace.

Each vial with its contents was stored at 55° C. for four weeks and was analyzed (after 2 and 4 weeks of storage) for formation of valdecoxib (using HPLC), and for pH and color change. Data are shown in Tables 10 and 11.

TABLE 10
Stability of formulations of Example 8
after 2 weeks storage at 55° C.

| | | Valdec- | | |
|---|---|---|---|---|
| | | oxib | | |
| | Fill | formed | | |
| Formulation | volume (ml) | (%) | Appearance | pH |
| LF25 | 1.2 | 1.1 | Clear and colorless | 8.19 |
| LF26 | 1.2 | 1.0 | Clear, almost colorless | 8.34 |
| LF27 | 1.2 | 1.0 | Clear and colorless | 8.23 |
| LF28 | 1.2 | 1.0 | Clear, almost colorless | 8.26 |
| LF25 | 0.7 | 1.2 | Clear and colorless | 8.16 |
| LF26 | 0.7 | 1.0 | Clear and colorless | 8.25 |
| LF27 | 0.7 | 1.1 | Clear and colorless | 8.20 |
| LF28 | 0.7 | 1.0 | Clear and colorless | 8.33 |

TABLE 11
Stability of formulations of Example 8
after 4 weeks storage at 55° C.

| | | Valdec- | | |
|---|---|---|---|---|
| | Fill | oxib | | |
| | volume | formed | | |
| Formulation | (ml) | (%) | Appearance | pH |
| LF25 | 1.2 | 1.8 | — | 8.01 |
| LF26 | 1.2 | 1.7 | Clear, faint yellow | 8.24 |
| LF27 | 1.2 | 1.8 | Clear and colorless | 8.11 |
| LF28 | 1.2 | 1.7 | Clear, faint yellow | 8.26 |
| LF25 | 0.7 | 2.0 | Clear and colorless | 8.08 |
| LF26 | 0.7 | 1.7 | Clear, faint yellow | 8.16 |
| LF27 | 0.7 | 1.9 | Clear and colorless | 8.13 |
| LF28 | 0.7 | 1.7 | Clear, faint yellow | 8.13 |

These data show that each of formulations LF25-LF28 exhibited good parecoxib stability during storage for four weeks at 55° C. Furthermore, formulation LF27 at both fill volumes and formulation LF25 at the 0.7 ml fill volume remained clear and colorless through four weeks of storage.

What is claimed is:

1. A parenterally deliverable pharmaceutical composition comprising parecoxib in a form of a water soluble parecoxib salt, in dissolved and/or solubilized form in a solvent liquid that comprises water and one or more nonaqueous solubilizer(s) for valdecoxib that forms by conversion of parecoxib thereto, wherein the nonaqueous solubilizer(s) are substantially inert with respect to such conversion, such that upon storage of the composition in a closed container maintained at 55° C. for a period of 14 days, parecoxib constitutes at least about 95% of the total amount, expressed as parecoxib free acid equivalent, of parecoxib and valdecoxib in the composition, and wherein the composition has stabilizing means for inhibiting precipitation of parecoxib free acid.

2. The composition of claim 1 wherein the parecoxib salt is an alkali metal salt of parecoxib.

3. The composition of claim 1 wherein the parecoxib salt is parecoxib sodium.

4. The composition of claim 1 wherein the parecoxib is present in an amount, expressed as parecoxib free acid equivalent, of about 1 to about 400 mg/ml of the composition.

5. The composition of claim 1 wherein the parecoxib is present in an amount, expressed as parecoxib free acid equivalent, of about 10 to about 50 mg/ml of the composition.

6. The composition of claim 1 wherein the nonaqueous solubilizer(s) are selected from the group consisting of polyethylene glycol, ethanol and dimethylacetamnide.

7. The composition of claim 1 wherein the nonaqueous solubilizer(s) comprise polyethylene glycol.

8. The composition of claim 1 wherein the stabilizing means comprises a pH controlling means for maintaining pH of the composition not lower than about 7.4.

9. The composition of claim 8 wherein the pH controlling means comprises one or more buffer(s) as a component of the solvent liquid.

10. The composition of claim 9 wherein the one or more buffer(s) are selected from the group consisting of phosphate, 2-amino-2-(hydroxymethyl)-1,3-propanediol, ascorbate and maleate buffers.

11. The composition of claim 8 wherein the pH controlling means comprises polyethylene glycol as a component of the solvent liquid at a concentration therein of not less than about 50% by weight.

12. The composition of claim 1 wherein the stabilizing means comprises an oxygen limiting means for limiting effective exposure of the composition to oxygen.

13. The composition of claim 12 wherein the oxygen limiting means comprises one or more antioxidant(s) as a component of the solvent liquid.

14. The composition of claim 13 wherein the one or more antioxidant(s) are selected from the group consisting of butylated hydroxyanisole, ascorbate and methionine.

15. The composition of claim 13 having a total antioxidant amount of about 0.001% to about 5% by weight of the solvent liquid.

16. The composition of claim 12 wherein the oxygen limiting means comprises an oxygen-limited microatmosphere in contact with the composition.

17. A parenterally deliverable pharmaceutical composition comprising a parecoxib component in a form of a water soluble parecoxib salt, in dissolved and/or solubilized form in a solvent liquid that comprises (a) a water component, (b) a nonaqueous solubilizer component effective to solubilize valdecoxib that forms by conversion of parecoxib thereto, said nonaqueous solubilizer component being substantially inert with respect to such conversion, and (c) a parecoxib salt stabilizer component effective to inhibit precipitation of parecoxib free acid; said nonaqueous solubilizer and parecoxib salt stabilizer components being the same or different; wherein upon storage of the composition in a closed container maintained at 55° C. for a period of 14 days, parecoxib constitutes at least about 95% of the total amount, expressed as parecoxib free acid equivalent, of parecoxib and valdecoxib in the composition.

18. The composition of claim 17 wherein the parecoxib salt is an alkali metal salt of parecoxib.

19. The composition of claim 17 wherein the parecoxib salt is parecoxib sodium.

20. The composition of claim 17 wherein the parecoxib component is present in an amount, expressed as parecoxib free acid equivalent, of about 1 to about 400 mg/ml of the composition.

21. The composition of claim 17 wherein the parecoxib component is present in an amount, expressed as parecoxib free acid equivalent, of about 10 to about 50 mg/ml of the composition.

22. The composition of claim 17 wherein the nonaqueous solubilizer component comprises one or more solubilizer(s) selected from the group consisting of polyethylene glycol, ethanol and dimethylacetamide.

23. The composition of claim 17 wherein the nonaqueous solubilizer component comprises dimethylacetamide.

24. The composition of claim 23 wherein the dimethylacetamide is in an amount of about 0.01% to about 15% by weight of the solvent liquid.

25. The composition of claim 17 wherein the nonaqueous solubilizer component comprises ethanol.

26. The composition of claim 25 wherein the ethanol is present in an amount of about 1% to about 30% by weight of the solvent liquid.

27. The composition of claim 17 wherein the solvent liquid comprises polyethylene glycol.

28. The composition of claim 27 wherein the polyethylene glycol is at a concentration of about 20% to about 80% by weight of the solvent liquid.

29. The composition of claim 27 wherein the polyethylene glycol has an average molecular weight of about 200 to about 1000.

30. The composition of claim 27 wherein the polyethylene glycol has an average molecular weight of about 400 to about 800.

31. The composition of claim 17 wherein the parecoxib salt stabilizer component comprises one or more agent(s) selected from the group consisting of pH buffers, antioxidants, and polyethylene glycol at a polyethylene glycol concentration not less than about 50% by weight of the solvent liquid.

32. The composition of claim 17 wherein the parecoxib salt stabilizer component comprises one or more pH buffer(s) selected from the group consisting of phosphate, 2-amino-2-(hydroxymethyl)-1,3-propanediol, ascorbate and maleate buffers.

33. The composition of claim 32 wherein the parecoxib salt stabilizer component comprises one or more pH buffers at a total buffer concentration of about 1 to about 50 mM in the composition.

34. The composition of claim 17 wherein the parecoxib salt stabilizer component comprises one or more antioxidant(s) selected from the group consisting of butylated hydroxyanisole, ascorbate and methionine.

35. The composition of claim 17 wherein the parecoxib salt stabilizer component comprises one or more antioxidant(s) in a total antioxidant amount of about 0.001% to about 5% by weight of the solvent liquid.

36. The composition of claim 17 wherein the parecoxib salt stabilizer component comprises polyethylene glycol at a polyethylene glycol concentration not less than about 50% by weight of the solvent liquid.

37. The composition of claim 36 wherein the polyethylene concentration is about 55% to about 75% by weight of the solvent liquid.

38. The composition of claim 36 wherein the polyethylene glycol has an average molecular weight of about 200 to about 1000.

39. The composition of claim 36 wherein the polyethylene glycol has an average molecular weight of about 400 to about 800.

40. A parenterally deliverable pharmaceutical composition comprising a solvent liquid having dissolved and/or solubilized therein a water soluble parecoxib salt in an amount, expressed as parecoxib free acid equivalent, of about 10 to about 50 mg/ml of the composition, wherein the solvent liquid comprises (a) water; (b) polyethylene glycol having an average molecular weight of about 400 to about 800 in an amount of about 30% to about 70% by weight of the solvent liquid; and (c) at least one of (i) butylated hydroxyanisole in an amount of about 0.001% to about 1% by weight of the solvent liquid and (ii) one or more buffers selected from the group consisting of 2-amino-2-(hydroxymethyl)-1,3-propanediol, maleate, ascorbate and phosphate buffers, in a total buffer concentration of about 1 to about 50 mM in the composition.

41. A parenterally deliverable pharmaceutical composition comprising a solvent liquid having dissolved and/or solubilized therein a water soluble parecoxib salt in an amount, expressed as parecoxib free acid equivalent, of about 10 to about 50 mg/ml of the composition, wherein the solvent liquid comprises (a) water; and (b) polyethylene glycol having an average molecular weight of about 400 to about 800 in an amount of about 55% to about 75% by weight of the solvent liquid.

42. The composition of claim 41 wherein the parecoxib salt is parecoxib sodium in an amount, expressed as parecoxib free acid equivalent, of about 40 mg/ml of the composition, and wherein the solvent liquid comprises polyethylene glycol having an average molecular weight of about 600 in an amount of about 65% by weight of the solvent liquid.

43. An article of manufacture comprising the composition of claim 17 in a sealed container.

44. The article of claim 43 wherein the sealed container is selected from the group consisting of a vial, an ampoule, a syringe, a packet, a pouch, and an auto-injector.

45. The article of claim 43 wherein the container has an interior comprising a fill volume occupied by the composition and a headspace volume occupied by an oxygen limited microatmosphere.

46. The article of claim 45 wherein the microatmosphere consists essentially of one or more inert gases selected from the group consisting of noble gases and nitrogen.

47. The article of claim 45 wherein the ratio of fill volume to headspace volume is not less than about 1:5.

48. The article of claim 45 wherein the headspace volume has an oxygen pressure of not more than about 5%.

49. The article of claim 43 wherein the parecoxib salt is present at a suitable concentration for parenteral administration without further dilution.

50. The article of claim 43 wherein the parecoxib salt is present in an amount corresponding to 1 to about 30 unit doses.

51. The article of claim 43 wherein the parecoxib salt is present in an amount corresponding to a single unit dose.

52. The article of claim 51 wherein the sealed container is a syringe.

53. The article of claim 48 wherein the ratio of fill volume to headspace volume is not less than about 1:5, and wherein the parecoxib salt is present at a suitable concentration for parenteral administration without further dilution and in a total amount corresponding to 1 to about 30 unit doses.

54. An article of manufacture comprising the pharmaceutical composition of claim 40 in a sealed container, wherein the container has an interior comprising a fill volume occupied by the composition and a headspace volume occupied by a microatmosphere having an oxygen pressure of not more than about 5%, wherein the ratio of fill volume to headspace volume is not less than about 1:5, and wherein the parecoxib salt is present in a total amount corresponding to 1 to about 30 unit doses.

55. An article of manufacture comprising the pharmaceutical composition of claim 41 in a sealed container, wherein the container has an interior comprising a fill volume occupied by the composition and a headspace volume occupied by a microatmosphere having an oxygen pressure of not more than about 5%, wherein the ratio of fill volume to headspace volume is not less than about 1:5, and wherein the parecoxib sodium is present in a total amount corresponding to 1 to about 30 unit doses.

56. A method of treating a subject having a condition or disorder wherein treatment with a COX-2 inhibitory drug is indicated, the method comprising parenterally administering a therapeutically effective amount of the composition of claim 1.

57. A method of treating a subject having a condition or disorder wherein treatment with a COX-2 inhibitory drug is indicated, the method comprising parenterally administering a therapeutically effective amount of the composition of claim 17.

58. A process for preparing a parenterally deliverable pharmaceutical composition, the process comprising a step of combining in any order, with mixing, (a) a parecoxib component in a form of a water soluble parecoxib salt; (b) a water component; (c) a nonaqueous solubilizer component effective to solubilize valdecoxib that forms by conversion of parecoxib thereto, said nonaqueous solubilizer component being substantially inert with respect to such conversion; and (d) a parecoxib salt stabilizer component effective to inhibit precipitation of parecoxib free acid; said nonaqueous solubilizer and parecoxib salt stabilizer components being the same or different; said water, nonaqueous solubilizer and parecoxib salt stabilizer components forming when mixed a solvent liquid wherein the parecoxib component is dissolved and/or solubilized; wherein upon storage of the composition in a closed container maintained at 55° C. for a period of 14 days, parecoxib constitutes at least about 95% by weight of the total amount, expressed as parecoxib free acid equivalent, of parecoxib and valdecoxib in the composition.

59. The composition of claim 58 wherein the parecoxib salt stabilizer component comprises one or more stabilizing agents selected from the group consisting of pH buffers, antioxidants, and polyethylene glycol in an amount providing a polyethylene glycol concentration not less than about 50% by weight of the composition.

60. The process of claim 58 wherein at least one of the nonaqueous solubilizer component and the parecoxib salt stabilizer component comprises polyethylene glycol.

61. The process of claim 60 wherein the polyethylene glycol has a peroxide content not greater than about 1.5 meq/kg.

62. The process of claim 58, further comprising a step of placing the composition in a sealed container.

63. A composition produced by the process of claim 58.
